(19) United States
(12) Patent Application Publication
(10) Pub. No.: US 2008/0027520 A1
(43) Pub. Date: Jan. 31, 2008

(54) LASER TREATMENT OF TISSUE

(75) Inventors: George Yoseung Choi, Redwood City, CA (US); Kasey Kai-Chi Li, Palo Alto, CA (US)

Correspondence Address: LEVINE BAGADE HAN LLP, 2483 EAST BAYSHORE ROAD, SUITE 100, PALO ALTO, CA, 94303, US

(73) Assignee: Zoom Therapeutics, Inc., East Palo Alto, CA (US)

(21) Appl. No.: 11/750,873

(22) Filed: May 18, 2007

Related U.S. Application Data
(60) Provisional application No. 60/820,322, filed on Jul. 25, 2006.
(60) Provisional application No. 60/820,328, filed on Jul. 25, 2006.
(60) Provisional application No. 60/863,018, filed on Oct. 26, 2006.

(51) Int. Cl. A61N 5/067; A61N 7/02

(52) U.S. Cl. 607/89; 601/2

(57) ABSTRACT

Laser treatment of tissue, particularly the tissues in or around the nasal and oral cavities, are described herein. One method for reducing the size of the tissue being treated is to apply laser energy to the underlying tissue. One instrument may be used to deliver laser energy and to optionally provide an infusion or injection of a fluid directly into the tissue as well as optionally provide for ultrasound energy application as well. One or more optical fibers which may extend through needles inserted into the tissue may be utilized to deliver the laser energy.

### CROSS-REFERENCE TO RELATED APPLICATIONS

This application claims the benefit of priority to the following U.S. Provisional Patent Application Nos. 60/820,322 and 60/820,328 both filed Jul. 25, 2006; and 60/863,018 filed Oct. 26, 2006, each of which is incorporated herein by reference in its entirety.

### TECHNICAL FIELD OF THE INVENTION

The present invention relates to devices and methods for treating soft tissue regions for clearing or reducing tissue obstructions. More particularly, the present invention relates to laser devices and methods for clearing obstructed tissue regions by treating areas within the tissue.

### BACKGROUND OF THE INVENTION

Treatments for chronically obstructed airway passages of a patient vary greatly. They typically range from the administration of medications to surgical interventional procedures. Examples of typical medication include such types as protriptyline, medroxyprogesterone, acetazolamide, theophylline, nicotine, and other medications. Although helpful at times, they are rarely completely effective. Moreover, such medications frequently have undesirable side effects.

Examples of typical surgical interventions include uvulopalatopharyngoplasty, tonsillectomy, surgery to correct severe retrognathia, and tracheostomy. Other surgical procedures include pulling the tongue as forward as possible and surgically cutting and removing sections of the tongue and other structures which can close off the upper airway passage. These procedures may be effective but the risk of surgery in these patients can be prohibitive and the procedures are often unacceptable to the patients.

As shown in FIG. 1, the sinus cavity 10 which can become obstructed include the nasal passageways leading from the nose 12 to the pharynx 16. The nasal airway has several compartments, namely the inferior 18, middle 20, and superior nasal meatus 22. The turbinates, also referred to as nasal concha, are a series of tissues which form at least a portion of these nasal compartments 18, 20, 22. Forming a portion of the inferior nasal meatus 18 is the inferior nasal turbinate 24. The inferior 24 and middle nasal turbinate 26 each form a portion of the middle nasal meatus 20. The middle 26 and superior nasal turbinate 28 each form a portion of the superior nasal meatus 22. When the inferior 24, middle 26 and/or superior nasal turbinate 28 become enlarged, the various nasal meatus which allow air to pass through the nostril 14 into the pharynx 16 can become obstructed.

Pharmaceuticals such as anti-histamines and anti-inflammatory drugs have been developed for reducing the size of the turbinates. However, pharmaceuticals are not always completely efficacious and generally do not provide a permanent reduction in turbinate size. In addition, pharmaceuticals can have adverse side effects.

Opening of obstructed nasal airways 18, 20, 22 by reducing the size of the turbinates 24, 26, 28 has been performed using surgical and pharmaceutical treatments. Such surgical procedures include anterior and posterior ethmoidectomy, an example of which is a procedure known as the Wigand procedure which involves transecting a portion of the middle turbinate 26. Other procedures have included inserting an electro-surgical probe, such as a radio-frequency (RF) energy probe, directly into a portion of the inferior turbinate 24. Once inserted, RF energy is applied to ablate the tissue interior of the turbinate 24. However, complications, such as excessive hemorrhaging, infection, perforation, scarring, adhesion of the turbinate, and intra-operative and post-operative pain may be present.

Accordingly, there exists a need for devices and methods which are efficacious and safe in clearing obstructed nasal passageways, at least for an extended period of time.

### SUMMARY OF THE INVENTION

By reducing the size of a nasal turbinate, particularly the inferior nasal turbinate, obstruction of a nasal meatus such as the inferior nasal meatus can be reduced thereby improving the air flow through the nasal meatus. One method for reducing the size of the inferior nasal turbinate involves applying ultrasound energy to the tissue regions beneath the surface of the inferior turbinate. Ultrasound energy may be particularly advantageous in damaging the tissues beneath the turbinate surface layer by enabling the delivery of energy to a predetermined distance through the tissue without damaging the tissue surface while injuring the underlying tissue to create scarring. Moreover, because ultrasound energy may leave the turbinate tissue surface undisturbed, the need for surgical cutting is obviated.

One variation of a treatment instrument which may be used to deliver ultrasound energy to the underlying turbinate tissue may also be configured to provide an infusion or injection of a fluid directly into the turbinate being treated by the ultrasound energy. The fluid injected into the turbinate may be used to bulk up the physical size of the turbinate by injecting the fluid to present a larger surface area to the ultrasound transducers positioned along the instrument. The enlarged surface area may help to ensure that the ultrasound energy is properly delivered directly into the intended turbinate tissue rather than surrounding tissues.

The injected fluid may also be used for drug delivery directly into the treated turbinate tissue. For instance, anesthetic fluids or other fluids infused with analgesics may be injected into the turbinate tissue to provide for pain management during and after the application of the ultrasound energy. Additionally, other drugs for injection may include any number of medications, such as non-steroidal drugs, anti-inflammatory drugs, anti-bacterial drugs, etc. which may be injected to control excessive post-operative swelling as well as infection. Additionally, the one or more injection needles may be utilized as a positioning tool for ensuring that the ultrasound energy, which is directional, is delivered into the intended turbinate tissue. For example, the injection needle(s) may be initially positioned directly within the turbinate tissue prior to application of the ultrasound energy since the ultrasound transducer(s) along the probe may be aligned with the injection needle(s). Accordingly, if the needle(s) is positioned directly within the turbinate tissue to be treated, the operator may be assured that the ultrasound energy will be directionally aligned with the appropriate turbinate tissue region.

The ultrasound and infusion probe may have an elongate shaft which is sufficient to allow for insertion and advancement into the nasal cavity and against the appropriate turbinate tissue surface. The distal end portion may be angled relative to the elongate shaft or it may be straight depending upon the desired configuration. The distal end portion may have an end effector assembly which has one or more hollow infusion/injection needles which are retractably disposed within the distal end portion. During advancement into the nasal cavity and positioning against the turbinate tissue, the infusion/injection needles may be positioned within the distal end portion so as to present a smooth atraumatic surface to the tissue. When a fluid is to be injected into the tissue after the probe has been desirably positioned against the tissue surface, a control or advancement mechanism on handle, which is connected to a proximal end of the shaft, may be actuated to advance the needles at least partially out of the distal end portion. Between or adjacent to the needles are one or more ultrasound transducers along the body of the distal end portion.

An electronic/fluid cable is electrically and fluidly connected to the handle and is further connected to a power/infusion assembly, which may hold a fluid reservoir and a pump electrically coupled to a controller or central processor. Any of the above-mentioned fluids, e.g., analgesics, anesthetics, anti-inflammatory drugs, water, saline, etc., may be filled within the reservoir for delivery through the cable and through the one or more infusion/injection needles for delivery into the turbinate tissue.

In use, the elongate shaft and distal end portion may be advanced through the patient's nostril and through the inferior nasal meatus against the tissue surface of the inferior nasal turbinate. The distal end portion of the elongate shaft may be positioned anywhere against the inferior nasal turbinate and the infusion/injection needles may be deployed from the distal end portion and pierced into the turbinate tissue, where the fluid may be injected and/or infused from the needles into the turbinate. As the fluid is injected into the tissue, the infused inferior turbinate may begin to expand in size thereby pressing against the distal end portion. The fluid may be stopped and the focused ultrasound energy may then be transmitted from the transducers into the underlying expanded turbinate tissue.

Once the injection and ultrasound treatment has been concluded, the damaged underlying turbinate tissue may scar and eventually reduce a size of the inferior turbinate, thereby resulting in an unobstructed inferior nasal meatus. The treatments may be performed periodically between extended time periods while the turbinate tissue regenerates or on an as-needed basis.

In alternative configurations, the distal end effectors may include a mechanism for securely pressing the surface of the elongate shaft against the turbinate tissue surface to be treated to ensure piercing of the needles into the tissue as well as sufficient contact for the ultrasound transmission. For instance, expandable balloons and wires or ribbon members which may be reconfigured from a low-profile configuration against the elongate shaft to an expanded shape may be utilized.

Moreover, the ultrasound and infusion probe may optionally include an additional radio-frequency energy generator to deliver RF energy to one or more needles to ablate the pierced tissue. The ultrasound and infusion probe may also optionally include a cooling unit fluidly connected via a fluid line to the power/infusion assembly. Cooled fluid may be fluidly connected through the elongate shaft to a cooling fluid port positioned along the distal end portion.

Additionally, aside from the use of ultrasound transducers for delivering energy to the turbinate tissue, laser energy may alternatively be used to facilitate turbinate tissue reduction while achieving hemostasis and minimizing tissue injury to surrounding tissue regions.

### BRIEF DESCRIPTION OF THE DRAWINGS

FIG. 1 shows an illustrative view of a nasal cavity and the passageways formed by the turbinates.

FIG. 2 shows a variation of a treatment instrument which may be used to deliver ultrasound energy as well as for providing an infusion or injection of a fluid directly into the turbinate being treated by the ultrasound energy.

FIGS. 3A and 3B illustrate partial cross-sectional detail views of a distal end portion of the elongate shaft showing the infusion/injection needles positioned within and projected out from the elongate shaft, respectively.

FIG. 3C shows another variation of an elongate shaft having an infusion/injection needle retractably positioned on a distal end of the shaft and with one or more ultrasound transducers also on the distal end.

FIG. 3D shows another variation of an elongate shaft having an additional tissue engaging tip on the distal end.

FIG. 3E shows yet another variation of an elongate shaft having a combination of infusion/injection needles along the length and distal end of the device.

FIG. 4A shows an elongate shaft advanced through the inferior nasal meatus for treating the inferior nasal turbinate.

FIG. 4B shows alternative positions for placing the elongate shaft against the turbinate to be treated.

FIG. 4C shows an elongate shaft advanced through the nostril for treating an anterior portion of the inferior nasal turbinate.

FIGS. 5A to 5C illustrate one method for infusing or injecting the fluid into the inferior turbinate and applying ultrasound energy to the expanded tissue and the resulting unobstructed inferior nasal meatus.

FIG. 6 illustrates an alternative variation where a single needle may be utilized with one or two ultrasound transducers.

FIG. 7 illustrates yet another alternative variation where three or more needles may be utilized with at least two ultrasound transducers in an alternating manner.

FIGS. 8A and 8B show variations for positioning of the needles and transducers relative to one another.

FIGS. 9A to 9C show side and end views, respectively, of one variation of a distal end portion which may be configured to include an expandable balloon.

FIGS. 10A and 10B show side and end views, respectively, of another variation of a distal end portion which may be configured to include a reconfigurable wire or ribbon member.

FIG. 11 shows an alternative configuration of the ultrasound and infusion assembly which may optionally utilize an RF generator and/or an optional cooling fluid reservoir assembly.

FIGS. 12A and 12B show top and side views, respectively, of an alternative ultrasound and infusion probe which may be configured to have a plurality of ultrasound transducers.

FIGS. 13A and 13B show side views of examples of an elongate shaft which is malleable or has at least a malleable portion.

FIG. 14 shows another variation used to deliver laser energy as well as for providing an infusion or injection of a fluid directly into the turbinate being treated.

FIGS. 15A and 15B show yet another variation where optical fibers may be placed through the elongate shaft and optionally advanced through the one or more needles.

FIG. 15C shows another variation where the shaft itself is tapered into a sharpened distal end with a lumen defined therethrough.

FIG. 16A shows a detail cross-sectional view of an advanceable needle defining a central lumen through which an optical fiber may be positioned.

FIG. 16B shows a detail cross-sectional view of another variation where a lumen opening may terminate along a surface of the needle proximal to a piercing tip.

FIG. 16C shows yet another variation where the optical fiber may be positioned along an outer surface of the piercing needle.

FIGS. 17A to 17C illustrate one method in which an infusion needle may be advanced into the tissue to be treated to infuse a fluid and then an optical fiber may be advanced through the needle to treat the region of tissue via laser energy.

FIG. 18A illustrates another method of treatment where the system may be used to treat other body regions in the patient, such as the soft palate.

FIG. 18B illustrates another method for treating the pharyngeal tissues.

FIG. 18C illustrates yet another method for treating the tissues around the base of the patient's tongue.

### DETAILED DESCRIPTION OF THE INVENTION

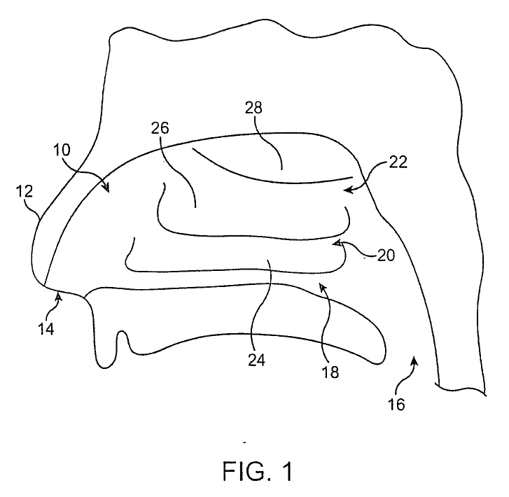

As described above in FIG. 1, connecting the nostril 14 and pharynx 16 are the passageways of the inferior nasal meatus 18, the middle nasal meatus 20, and the superior nasal meatus 22. Forming at least a portion of each of these passageways are the nasal turbinates. Forming at least a portion of the inferior nasal meatus 18 is the inferior nasal turbinate 24. Forming at least a portion of the middle nasal meatus 20 is the inferior nasal turbinate 24 and the middle nasal turbinate 26. Forming at least a portion of the superior nasal meatus 22 is the middle nasal turbinate 26 and the superior nasal turbinate 28.

By reducing the size of a nasal turbinate, particularly the inferior nasal turbinate 24, obstruction of a nasal meatus such as the inferior nasal meatus 18 can be reduced. By reducing an obstruction of a nasal meatus, air flow through the nasal meatus is improved. One method for reducing the size of the inferior nasal turbinate 24 involves the application of ultrasound energy to the tissue regions beneath the surface of the inferior turbinate 24. Ultrasound energy may be particularly advantageous in damaging the tissues beneath the turbinate surface layer by enabling the delivery of energy to a predetermined distance through the tissue without damaging the tissue surface while injuring the underlying tissue to create scarring. Moreover, because ultrasound energy may leave the turbinate tissue surface undisturbed, the need for surgical cutting is obviated. The affected targeted tissue may scar and atrophy and eventually shrink and/or prevent the enlargement of the turbinate 24.

Although reference is made particularly to treatment of the inferior turbinate 24, this is done so for illustrative purposes. The procedures and devices described herein may easily be applied to any of the nasal turbinates 24, 26, 28 and are intended to be so.

However, because the size of the turbinate to be treated may vary greatly between patients, there is variability in the application of ultrasound energy that an ultrasound energy delivery device needs to compensate for. Additionally, even the application of ultrasound energy may produce pain and discomfort in the patient being treated due to the highly vascularized structure of the turbinates.

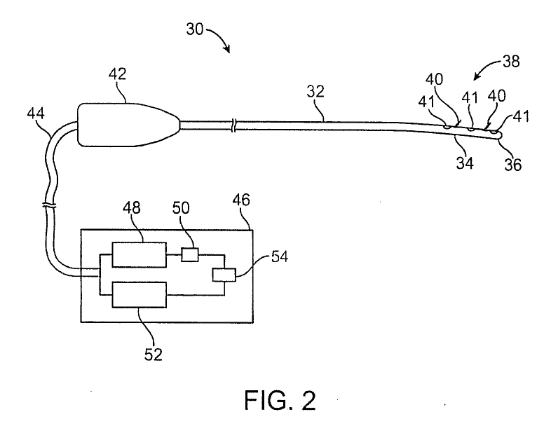

FIG. 2 illustrates a variation of a treatment instrument which may be used to deliver ultrasound energy for treating the tissues underlying the turbinate surface as well as for providing an infusion or injection of a fluid directly into the turbinate being treated by the ultrasound energy. The fluid injected into the turbinate may serve a number of different purposes. One purpose is to bulk up the physical size of the turbinate by injecting the fluid to present a larger surface area to the ultrasound transducers positioned along the instrument. The enlarged surface area may help to ensure that the ultrasound energy is properly delivered directly into the intended turbinate tissue rather than surrounding tissues. Examples of fluids which may be used for bulking the turbinate tissue may include any number of suitable fluids, e.g., saline, water, etc.

Another purpose is for drug delivery directly into the treated turbinate tissue. For instance, anesthetic fluids or other fluids infused with analgesics (e.g., lidocaine with or without epinephrine, marcaine with or without epinephrine, etc.) may be injected into the turbinate tissue to provide for pain management during and after the application of the ultrasound energy. Additionally, other drugs for injection may include any number of medications, such as steroidal drugs (e.g., corticosteroids, dexamethasone, beclomethasone, etc.), non-steroidal drugs (e.g., non-steroidal anti-inflammatory drugs, etc.), anti-inflammatory drugs, anti-histamines (e.g., diphenhydramine, etc.), anti-bacterial drugs, etc. which may be injected to control excessive post-operative swelling as well as infection.

Yet another purpose may be to utilize the one or more injection needles as a positioning tool for ensuring that the ultrasound energy, which is directional, is delivered into the intended turbinate tissue. For example, the injection needle(s) may be initially positioned directly within the turbinate tissue prior to application of the ultrasound energy since the ultrasound transducer(s) along the probe may be aligned with the injection needle(s). Accordingly, if the needle(s) is positioned directly within the turbinate tissue to be treated, the operator may be assured that the ultrasound energy will be directionally aligned with the appropriate turbinate tissue region.

Returning now to FIG. 2, ultrasound and infusion probe 30 is illustrated as having an elongate shaft 32 with a distal end portion 34 having a rounded or blunted atraumatic tip 36 to prevent trauma to contacted tissue. Elongate shaft 32 may have a length which is sufficient to enable the insertion of distal end portion 34 into the nasal cavity of a patient. Accordingly, the length of shaft 32 may range anywhere from several centimeters to 25 cm or longer while the distal end portion may range anywhere, e.g., from 10 to 30 mm in length or longer if so desired. The elongate shaft 32 itself may have conform to any cross-sectional area so long as the overall size is sufficient to allow for insertion and advancement into the nasal cavity and against the appropriate turbinate tissue surface. However, elongate shaft 32 may be typically circular with a diameter ranging anywhere from 4 to 5 mm or more. Moreover, elongate shaft 32 may optionally define one or more visual markings or indicators along its length to indicate a depth of the shaft 32 into the nasal cavity by comparison against the patient nostril 14.

The distal end portion 34 may be angled relative to the elongate shaft 32 or it may be straight depending upon the desired configuration. The distal end portion 34 may have an end effector assembly 38 which has one or more hollow infusion/injection needles 40 which are retractably disposed within the distal end portion 34. During advancement into the nasal cavity and positioning against the turbinate tissue, the infusion/injection needles 40 may be positioned within the distal end portion 34 so as to present a smooth atraumatic surface to the tissue. When a fluid is to be injected into the tissue after the probe 30 has been desirably positioned against the tissue surface, a control or advancement mechanism on handle 42, which is connected to a proximal end of shaft 32, may be actuated to advance needles 40 at least partially out of distal end portion 34.

The illustration of FIG. 2 shows two retractable infusion/injection needles 40; however, fewer or additional needles 40 may be utilized depending upon the desired results and procedure to be undertaken. Between or adjacent to needles 40 are positioned, one or more ultrasound transducers 41 along the body of distal end portion 34. The illustration shows three ultrasound transducers for delivering the ultrasound energy, but fewer or additional transducers 41 may be utilized or positioned along the distal end portion 34.

An electronic/fluid cable 44 is electrically and fluidly connected to handle 42 and is further connected to a power/infusion assembly 46. Within assembly 46 is a fluid reservoir 48 and a pump 50 electrically coupled to controller or central processor 54. Any of the above-mentioned fluids, e.g., analgesics, anesthetics, anti-inflammatory drugs, water, saline, etc., may be filled within reservoir 48 for delivery through cable 44, elongate shaft 32 and through the one or more infusion/injection needles 40 for delivery into the turbinate tissue. The infusion rate of the fluid and control of the pump 50 may be determined by the controller 54. An example of a pump which is pre-programmed to inject a fluid in a controlled injection rate and which may be utilized with the pump 50 is commercially available as the CompuDent® delivery system and Wand® handpiece (Milestone Scientific, Inc., South Orange Livingston, N.J.). Power supply 52 may also be provided within assembly 46 and may be controlled by controller 54 to control the amount of energy provided by the ultrasound transducers 41 located in distal end portion 34.

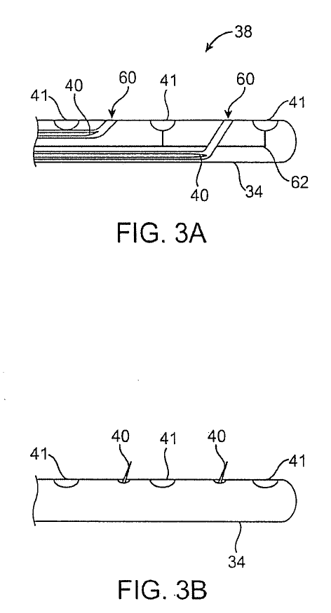

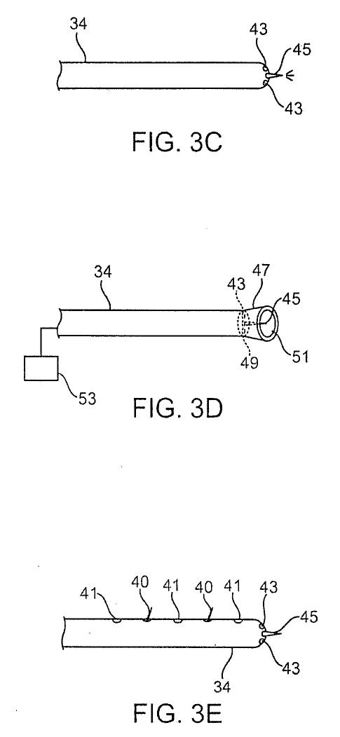

As mentioned above, during delivery and positioning of elongate shaft 32 against the turbinate tissue, the one or more needles 40 may be retracted within distal end portion 34, as shown in the partial cross-sectional detail view of FIG. 3A. As illustrated, infusion/injection needles 40 may be positioned within their respective needle lumens 60 positioned between the ultrasound transducers 41. The piezoelectric transducers of each of the ultrasound transducers 41 may be electrically coupled via wires 62 routed through elongate shaft 32 to the power supply 52 located within assembly 46. The piezoelectric transducer may be vibrated over a range of frequencies, e.g., anywhere from 0.5 to 12 MHz, or more typically between 5 to 12 MHz, to generate the ultrasound energy to treat the turbinate tissue.

When the infusion/injection needles 40 are to be deployed into or against the turbinate tissue, they may be advanced distally through needle lumens 60 until they project from a surface of the elongate shaft 32, as shown in FIG. 3B. Needles 40 may be configured to project from shaft 32 from less than 1 mm to more than 2 mm or anywhere therebetween provided that needles 40 are able to sufficiently contact against and/or into the turbinate tissue surface to inject the fluid.

In another variation, FIG. 3C illustrates the distal end portion 34 of elongate shaft 32 having at least one infusion/injection needle 45 retractably disposed at the distal tip. Also located at the tip are one or more ultrasound transducers 43 positioned adjacent to the retractable needle 45. Such a variation may be particularly useful for treating anterior portions of turbinate tissue.

FIG. 3D shows yet another variation in which the distal tip of distal end portion 34 further includes a tissue engaging hood 47 protruding distally from shaft 32. Hood 47 may be a removable or integrated tapered structure defining an opening 51 in communication with a vacuum lumen 49, which may be in fluid communication with a vacuum pump 53. Retractable needle 45 may be deployable to project into and/or through the opening 51 for contacting any turbinate tissue engaged therewith. In use, tissue engaging hood 47 may be positioned proximate or adjacent to a tissue region to be treated and a vacuum force through lumen 49 may be activated to securely draw the tissue therein. Once the drawn-in tissue is secured within engaging hood 47 by the vacuum, needle 45 may be projected into the secured tissue for injecting any fluids for treatment. Moreover, one or more ultrasound transducers 43 may also be positioned within the opening 51 to further treat the vacuum-secured tissue via ultrasound energy, as described herein. Once treatment has been completed, the vacuum may be de-activated to disengage the tissue.

FIG. 3E shows yet another variation which combines the ultrasound transducers 41 and retractable infusion/injection needles 40 which project along the length of the elongate shaft 32, as shown in FIG. 3B, with the distally disposed ultrasound transducers 43 and retractable infusion/injection needle 45 which projects from the distal tip of the shaft. This particular variation may be utilized to treat all aspects of the turbinate tissue, including the anterior and lateral portions of the tissue.

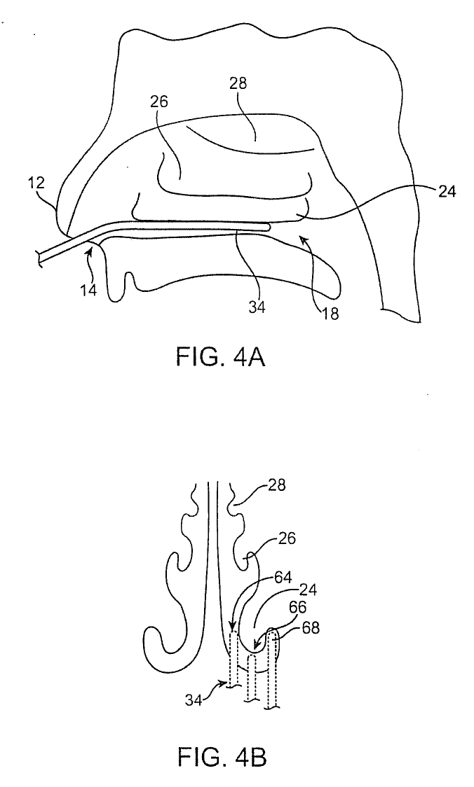

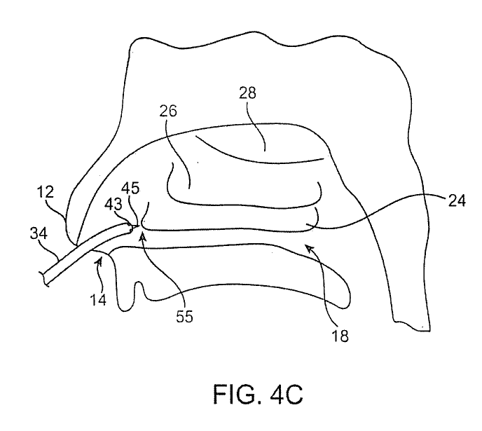

In use, elongate shaft 32 and distal end portion 34 may be advanced through the patient's nostril 14 and through the inferior nasal meatus 18 against the tissue surface of the inferior nasal turbinate 24, as shown in FIG. 4A. Distal end portion 34 of elongate shaft 32 may be positioned anywhere against the inferior nasal turbinate 24 at a first lateral surface 64, against an inferior surface 66, at a second lateral surface 68, or any or all three positions of the inferior turbinate 24, as shown in the end view of the turbinates 24, 26, 28 in FIG. 4B.

The instrument variations shown and described above in FIGS. 3C to 3E may be utilized in particular for treating anterior portions of the turbinate tissues, as previously mentioned. As illustrated in FIG. 4C, the distal portion 34 of the shaft 32 may be advanced through the patient's nostril 14 and positioned adjacent to an anterior portion 55 of the turbinate tissue. The infusion/injection needle 45, which may optionally be retracted during advancement into the nasal cavity or fully deployed, may inserted into the anterior portion 55 to inject the fluids. During and after the injection of fluids, the one or more ultrasound transducers 43 may be activated on the distal tip of the shaft to treat the underlying tissue, as further described below. Once the treatment has been completed, the shaft may be removed or repositioned to another portion of tissue for treatment.

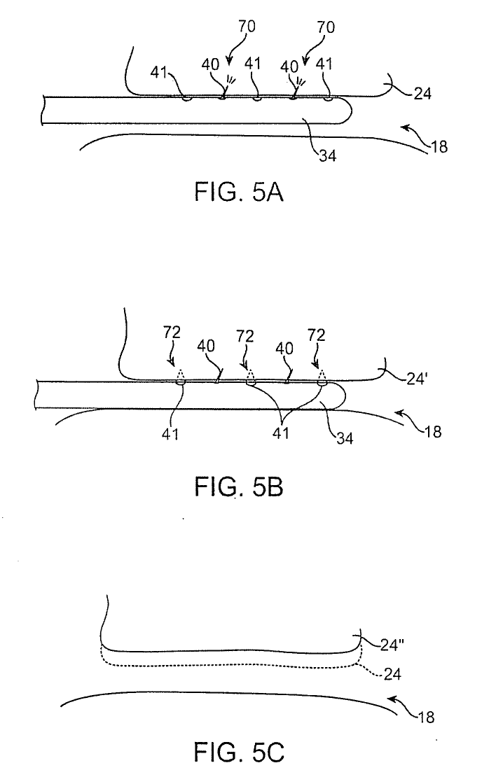

As described above and as illustrated in FIG. 5A, the infusion/injection needles 40 may be deployed from distal end portion 34 and pierced into the turbinate tissue 24, where the fluid 70 may be injected and/or infused from needles 40 into the turbinate 24. As the fluid is injected into the tissue, the infused inferior turbinate 24′ may begin to expand in size, as shown in FIG. 5B, thereby pressing against distal end portion 34. The fluid may be stopped and the focused ultrasound energy 72 may then be transmitted from transducers 41 into the underlying expanded turbinate tissue 24′. The ultrasound energy 72 may be applied anywhere from 1 second to 1 minute, and more particularly anywhere from 2 to 45 seconds and can be fired sequentially or simultaneously. Moreover, the focal point of the ultrasound energy 72 may range anywhere from about 1 mm or more away from the transducers 41 and more particularly anywhere from 2 to 4 mm away, so long as the focal point of the ultrasound energy 72 is able to be focused into the underlying turbinate tissue 24′ leaving the turbinate tissue surface unperturbed.

The increased size of the turbinate 24′ tissue surface presented to the transducers 41 may facilitate treatment of the underlying tissue as well as ensure that the appropriate tissue is treated. Moreover, once the ultrasound energy 72 has been applied at a first location, the needles 40 may be retracted and the distal end portion 34 may be moved to another region of the inferior turbinate 24′ to further effect treatment. Any amount of the expanded inferior turbinate 24′ may be treated, e.g., 3 to 4 cm of turbinate tissue along its length. With the infusion of anesthetics and/or anti-inflammatory drugs, any pain associated with the application of ultrasound energy and scarring of the tissue is eliminated or reduced.

Once the injection and ultrasound treatment has been concluded, the damaged underlying turbinate tissue may scar and eventually reduce a size of the inferior turbinate 24″, thereby resulting in an unobstructed inferior nasal meatus 18, as shown in FIG. 5C. The treatments may be performed periodically between extended time periods while the turbinate tissue 25″ regenerates or on an as-needed basis.

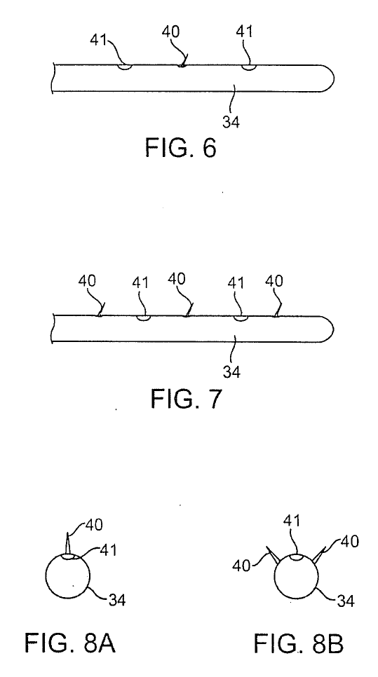

The configuration and number of infusion/injection needles 40 and ultrasound transducers 41 may be varied depending upon the desired effect. FIG. 6 illustrates an alternative variation where a single needle 40 may be utilized with one or two ultrasound transducers 41. Alternatively, FIG. 7 shows a variation where three or more needles 40 may be utilized with at least two ultrasound transducers 41 in an alternating manner. Moreover, the circumferential positioning of the needles 40 relative to the transducers 41 may also be varied. FIG. 8A shows one variation where each of the needles 40 and transducers 41 may be aligned linearly while FIG. 8B shows another variation where two or more needles 40 may be off-set to project at an angle relative to one another with the ultrasound transducer 41 positioned therebetween.

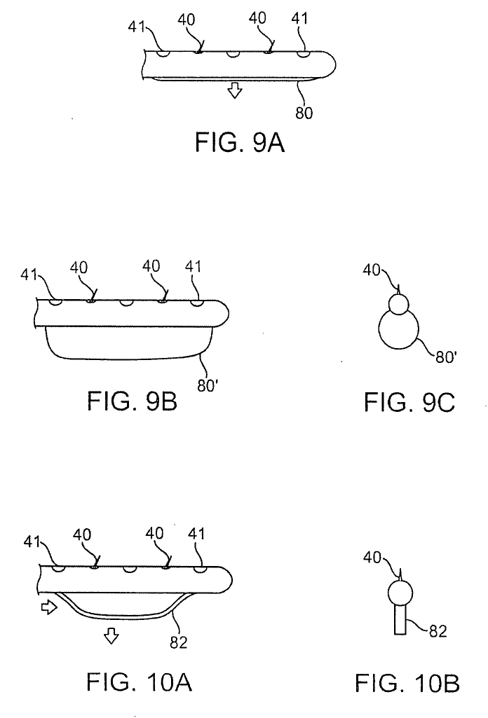

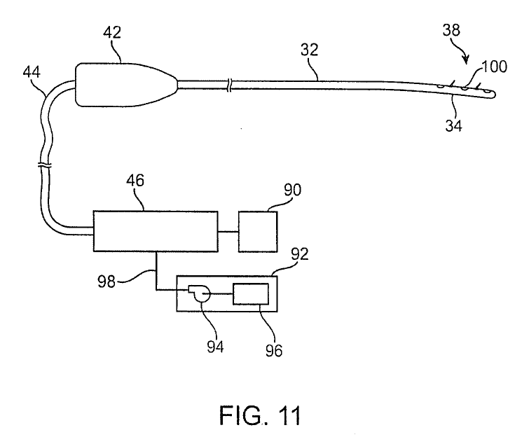

In alternative configurations, the distal end effectors may include a mechanism for securely pressing the surface of the elongate shaft against the turbinate tissue surface to be treated to ensure piercing of the needles into the tissue as well as sufficient contact for the ultrasound transmission. For instance, FIG. 9A illustrates one variation of a distal end portion which may be configured to include an expandable balloon 80. Once the shaft has been desirably positioned against the turbinate tissue surface, balloon 80′ may be expanded via a fluid such as water or saline or a gas such as air delivered through an inflation lumen defined through shaft 32, as shown in FIG. 9B and the end view in FIG. 9C. The expanded balloon 80′ may be utilized to press against the surrounding tissue within the inferior nasal meatus 18 to directionally press or force the shaft surface and needle 40 against or into the turbinate tissue. Once the desired treatment has been completed, balloon 80′ may be deflated and the elongate shaft 32 may be moved to another region of the turbinate or removed entirely.

Another variation of a mechanism is shown in the side and end views of FIGS. 10A and 10B, which illustrate a wire or ribbon member 82 which may be reconfigured from a low-profile configuration against the elongate shaft 32 to an expanded shape, as shown. When the elongate shaft 32 is to be securely presented against the tissue surface, wire or ribbon member 82 may be advanced or actuated from handle 42 to urge the member 82 into a reconfigured and expanded shape to push against the tissue.

In yet another configuration, the ultrasound and infusion probe 30 may optionally include an additional radio-frequency energy generator 90, which may be configured to deliver RF energy to one or more needles to ablate the pierced tissue. Ablation of the pierced regions of tissue may help to coagulate the pierced tissue. Moreover, the ultrasound and infusion probe 30 may also optionally include a cooling unit 92 fluidly connected via fluid line 98 to power/infusion assembly 46. Cooling unit 92 may comprise a pump 94 fluidly coupled to a reservoir 96 containing cooled or chilled fluid 96, e.g., saline, water, etc. The cooled fluid 96 may be fluidly connected through elongate shaft 32 to a cooling fluid port 100 positioned along distal end portion 100. Before, during, or after ultrasound energy transmission into the turbinate tissue, the cooled fluid may be pumped from reservoir 96 through cooling fluid port 100 to cool the surface of the turbinate tissue to ensure that the turbinate tissue surface is unperturbed by the energy applied beneath its surface.

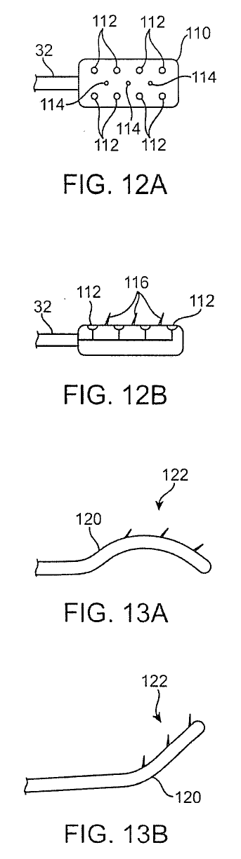

Other configurations for the ultrasound and infusion probe may be utilized. One example is shown in the top and side views of the ultrasound and infusion probe 110 shown in FIGS. 12A and 12B, respectively. In this configuration, a plurality of ultrasound transducers 112 may be positioned over a surface of the probe 110 and one or more needle openings 114 may be similarly positioned over the surface adjacent to the transducers 110. An example of a probe having multiple ultrasound transducers is shown in further detail in U.S. Pat. No. 6,361,531 to Hissong, which is incorporated herein by reference in its entirety. The one or more infusion/injection needles 116 may be deployed through the openings 114 when pressed against the turbinate tissue surface.

In any of the variations described herein, elongate shaft may be configured to be a malleable shaft 120, or at least have a distal portion which is malleable, from which the one or more infusion/injection needles 122 may be positioned. Such a malleable shaft may be configured by the user to conform to any number of configurations prior to advancement into the nasal cavity. For instance, the malleable shaft 120 may be configured into a curved configuration, as shown in FIG. 13A, or an angled configuration, as shown in FIG. 13B. In either case, once the procedure has been performed, the malleable shaft 120 may be reconfigured into yet another shape depending upon the desired configuration and anatomy of the patient.

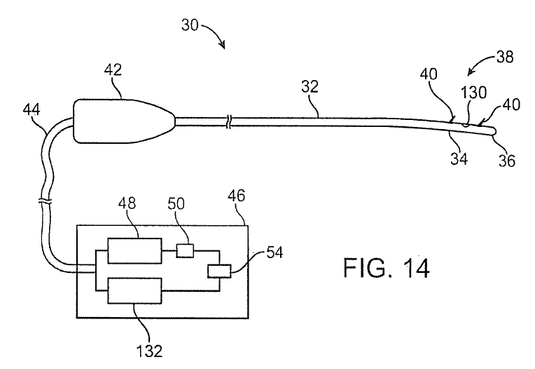

FIG. 14 shows another variation used to deliver laser energy as well as for providing an infusion or injection of a fluid directly into the turbinate being treated. The probe assembly may include a laser generator 132 for delivering laser energy through the probe shaft 32 to the distal end portion 34, e.g., via optical fibers positioned through the instrument with the terminal end of the optical fiber placed between or adjacent to the needles 40.

In use, the instrument may be delivered and positioned adjacent to the tissue to be treated. During or after the injection of the needles 40 and delivery of fluids in the tissue, the laser generator 132 may be actuated to deliver laser energy through the terminal end of the optical fiber 130. The laser may be configured as any number of laser instruments. For instance laser generator 132 may be an Argon laser or CO₂ laser capable of generating laser temperatures, e.g., of 750° to 900° C., to vaporize the underlying turbinate tissue.

Moreover, controller 54 may be configured to control laser generator 132 to deliver pulsed laser energy through fiber terminal end 130 for a controlled period of time and frequency.

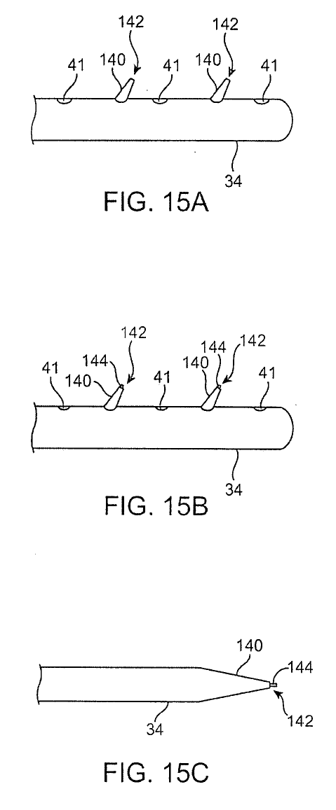

Another variation for delivering laser energy for tissue treatment is illustrated in the detail side views of FIGS. 15A and 15B. As shown, needles 140 may each define a lumen 142 therethrough, e.g., for infusion of fluids as described above, and/or advancing one or more corresponding optical fibers 144 directly into and through the needles 140, as shown in FIG. 15B. The optical fiber 144 may be advanced through the lumen 142 of the needle 140 after or during infusion of the fluid into the tissue. Alternatively, the infusion of fluids may be omitted entirely and the optical fiber 144 may be advanced through the needle 140 after or simultaneously with the needle 140 when projected from the shaft 34 into the tissue to be treated.

FIG. 15C shows another variation where the shaft 34 itself may taper into a sharpened distal end with lumen 142 defined therethrough. As such, the tapered end may function as a piercing needle through which optical fiber 144 may be passed through. The fluid infusion may also be infused through lumen 142 prior to, during, or after (or omitted entirely) placement of the optical fiber 144 within lumen 142 for treatment upon the tissue. The example illustrated above in FIG. 4C may optionally utilize this particular variation as well to effect laser tissue treatment.

Moreover, the laser energy passed through the optical fibers 142 may be utilized in conjunction with the ultrasound energy delivered via the one or more ultrasound transducers 41, as above, or alone. Furthermore, the optical fibers may be advanced through any of the needles described herein for laser treatment of the tissue and the use of ultrasound transducers may be omitted entirely as well.

In passing the optical fiber 144 through the needle body, the fiber 144 may be independently translatable within the needle lumen 142. In this variation, the fiber 144 may be passed through the same lumen utilized for fluid infusion through the needle, if fluid infusion is utilized. Alternatively, the optical fiber 144 may be affixed within the lumen 142 of the needle such that advancement or retraction of the needle also likewise advances or retracts the optical fiber 144 relative to the elongate shaft 34. Moreover, the optical fiber 144 in either case may be configured (if affixed) or otherwise urged (if translatable) to extend just proximal to, adjacent with, or distally beyond the lumen opening or needle tip and into the tissue during treatment.

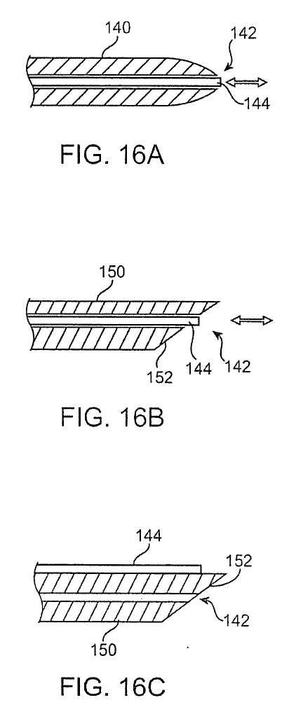

FIG. 16A illustrates one variation of optical fiber 144 positioned within lumen 142 of needle 140 in the partial cross-sectional detail view. Optical fiber 144 may be optionally translatable relative to needle 140, as indicated by the arrow, and positioned centrally through needle 140 such that the distal tip of optical fiber 144 is extendable through the distal tip of needle 140.

FIG. 16B illustrates another variation where optical fiber 144 may be optionally translatable, as indicated by the arrow, relative to needle 150 which defines an angled piercing surface 152. Optical fiber 144 may exit the lumen opening at a location along a side surface of the needle 50 proximal to the piercing tip so as not to inhibit entry of the needle 150 into the tissue to be treated.

FIG. 16C illustrates yet another variation where optical fiber 144 may be optionally placed or integrated along an outer surface of the needle 150 leaving lumen 142 open for fluid infusion. Optical fiber 144 may be adhered or affixed to needle 150 via any number of mechanisms, e.g., adhesives, outer sheath, etc., or otherwise integrated with the body of needle 150.

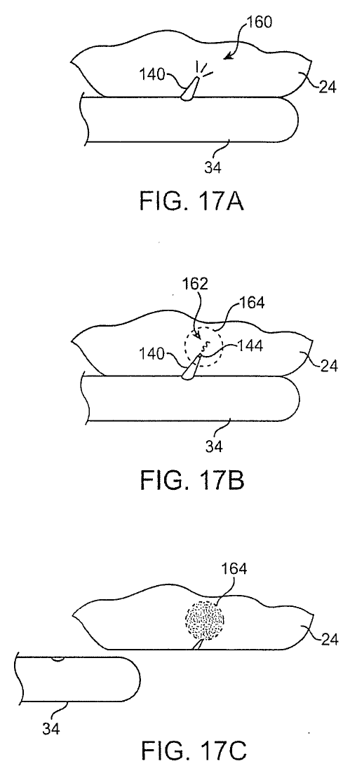

In an exemplary method of use, the elongate shaft 34 may be advanced with the needles in their retracted position within shaft 34 and placed against the region of tissue to be treated, e.g., the inferior nasal turbinate 24, as shown in FIG. 17A. One or more needles 140 (a single needle is shown for illustrative purposes only and is not intended to be limiting) may be advanced from shaft 34 and into the tissue. Once desirably positioned, fluid 160 may be optionally infused into the tissue, as described above, and optical fiber 144 may be advanced through needle 140. Alternatively, optical fiber 144 may be advanced into the tissue 24 simultaneously with needle 140 and infusion of the tissue may be omitted.

With optical fiber 144 positioned proximate to or within the tissue 24, laser energy 162 may be passed through optical fiber 144, as described above, to ablate the tissue region 164 around the needle 140, as shown in FIG. 17B. Optionally, ultrasound energy may be applied prior to, during, or after application of the laser energy depending upon the desired results. Once the treatment has been completed, needle 140 and optical fiber 144 may be retracted or withdrawn and elongate shaft 34 may be repositioned to another tissue region or withdrawn entirely from the patient body, as shown in FIG. 17C.

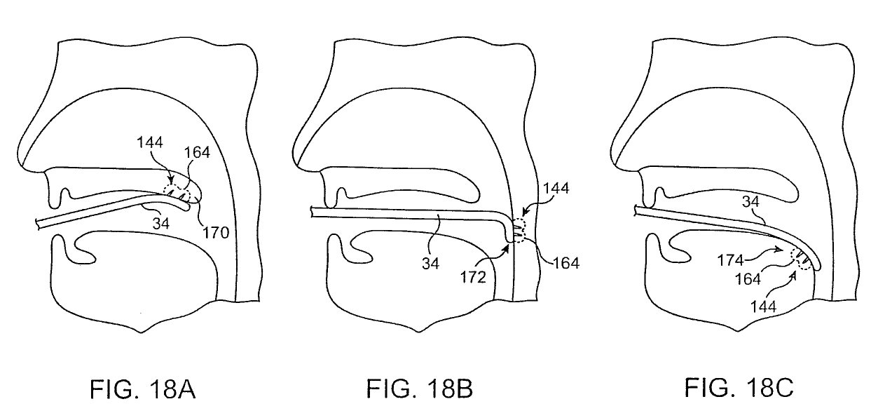

In alternative methods of use, other regions of the patient body may be treated with the system described herein. An example is illustrated in FIG. 18A where the elongate shaft 34 may be advanced into the patient's mouth and the one or more needles may be advanced into the soft palate 170 region. Optical fibers 144 may be utilized to treat the tissue region within or around the soft palate 170. In another example, elongate shaft 34 may be advanced further into the patient's mouth to treat areas in or around the pharyngeal tissue 172, as shown in FIG. 18B. In yet another example, the tissue regions in or around the base of the tongue 174 may be treated, as shown in FIG. 18C.

The applications of the devices and methods discussed above are not limited to the treatment of the tissue regions in or around the nasal and oral cavities but may include any number of further treatment applications. Other treatment sites may include areas or regions of the body such as soft tissue bodies. Modification of the above-described assemblies and methods for carrying out the invention, and variations of aspects of the invention that are obvious to those of skill in the art are intended to be within the scope of the claims.

What is claimed is:

1. An apparatus for treating tissues within a nasal cavity, comprising:
an elongate shaft having a distal end, a proximal end, and a length therebetween;
at least one optical fiber terminal end positioned near or at the distal end, wherein a proximal end of the optical fiber is in optical communication with a laser generator; and
at least one needle disposed near or at the distal end, wherein the at least one needle is retractably positioned to extend from a surface of the shaft.

2. The apparatus of claim 1 wherein the elongate shaft is sized to be advanced through a nostril and into a nasal meatus of the nasal cavity.

3. The apparatus of claim 1 wherein the elongate shaft is malleable.

4. The apparatus of claim 1 wherein the optical fiber terminal end is positioned adjacent to the at least one needle.

5. The apparatus of claim 1 further comprising a plurality of optical fiber terminal ends positioned near or at the distal end.

6. The apparatus of claim 1 wherein the at least one needle comprises a hollow infusion or injection needle.

7. The apparatus of claim 1 further comprising a plurality of needles disposed near or at the distal end, wherein the plurality of needles are retractably positioned to extend from the surface of the shaft.

8. The apparatus of claim 1 further comprising a handle assembly attached to the proximal end of the elongate shaft.

9. The apparatus of claim 1 further comprising a fluid reservoir in fluid communication with the at least one needle.

10. The apparatus of claim 1 further comprising a laser generator in optical communication with the optical fiber.

11. The apparatus of claim 1 further comprising an expandable member disposed near or at the distal end, wherein the expandable member is reconfigurable to an expanded configuration which urges the at least one needle against a tissue region of interest.

12. The apparatus of claim 1 further comprising a cooling fluid reservoir in fluid communication with at least one cooling port defined near or at the distal end.

13. A method of treating tissue within a nasal cavity, comprising:
positioning an elongate shaft having a distal end against a tissue region of interest within the nasal cavity;
piercing the tissue region via at least one needle retractably disposed near or at the distal end;
infusing or injecting a fluid through the at least one needle into the tissue region; and
applying laser energy to a surface of the tissue region via at least one optical fiber terminal end positioned near or at the distal end.

14. The method of claim 13 wherein positioning comprises advancing the elongate shaft through a nostril of a patient and through an inferior nasal meatus.

15. The method of claim 14 further comprising contacting the distal end against an inferior nasal turbinate.

16. The method of claim 13 wherein piercing comprises piercing the tissue region via a plurality of needles.

17. The method of claim 13 wherein piercing further comprises advancing the at least one needle from within the elongate shaft to project externally of a surface of the elongate shaft.

18. The method of claim 13 wherein infusing or injecting comprises infusing or injecting a fluid selected from the group consisting of anesthetics, analgesics, anti-inflammatory drugs, anti-histamines, non-steroidal drugs, steroidal drugs, anti-bacterial drugs, water, and saline.

19. The method of claim 13 wherein applying comprises transmitting laser energy via a plurality of optical fiber terminal ends positioned near or at the distal end.

20. The method of claim 13 further comprising applying a cooling fluid onto the surface of the tissue region.

21. The method of claim 13 further comprising urging the distal end against the tissue region of interest prior to applying laser energy against a surface.

22. An apparatus for treating tissues, comprising:
an elongate shaft having a distal end, a proximal end, and a length therebetween;
at least one needle disposed near or at the distal end, wherein the at least one needle is retractably positioned to extend from a surface of the shaft; and
at least one optical fiber positioned within or along the at least one needle such that a distal end of the optical fiber is movable with respect to the shaft.

23. The apparatus of claim 22 wherein the elongate shaft is malleable.

24. The apparatus of claim 22 wherein the at least one optical fiber is positioned within a lumen of the at least one needle.

25. The apparatus of claim 24 wherein the at least one optical fiber is translatably positioned within the lumen.

26. The apparatus of claim 24 wherein the at least one optical fiber is affixed with respect to the at least one needle.

27. The apparatus of claim 22 wherein the at least one needle comprises a hollow infusion or injection needle.

28. The apparatus of claim 22 further comprising a plurality of needles disposed near or at the distal end, wherein the plurality of needles are retractably positioned to extend from the surface of the shaft.

29. The apparatus of claim 22 further comprising a handle assembly attached to the proximal end of the elongate shaft.

30. The apparatus of claim 22 further comprising a fluid reservoir in fluid communication with the at least one needle.

31. The apparatus of claim 22 further comprising a laser generator in optical communication with a proximal end of the at least one optical fiber.

32. The apparatus of claim 22 further comprising an expandable member disposed near or at the distal end, wherein the expandable member is reconfigurable to an expanded configuration which urges the at least one needle against a tissue region of interest.

33. The apparatus of claim 22 further comprising a cooling fluid reservoir in fluid communication with at least one cooling port defined near or at the distal end.

34. A method of treating tissue via laser energy, comprising:
positioning an elongate shaft having a distal end against a tissue region of interest;
piercing the tissue region via at least one needle retractably disposed near or at the distal end;
advancing at least one optical fiber terminal end into the tissue region within or along the at least one needle; and
applying laser energy via the at least one optical fiber to the tissue region.

35. The method of claim 34 wherein positioning comprises advancing the elongate shaft through a nostril of a patient and against an inferior nasal turbinate.

36. The method of claim 34 wherein piercing comprises piercing the tissue region via a plurality of needles.

37. The method of claim 34 wherein piercing further comprises advancing the at least one needle from within the elongate shaft to project externally of a surface of the elongate shaft.

38. The method of claim 34 further comprising infusing or injecting a fluid through the at least one needle into the tissue region prior to applying laser energy.

39. The method of claim 38 wherein infusing or injecting comprises infusing or injecting a fluid selected from the group consisting of anesthetics, analgesics, anti-inflammatory drugs, anti-histamines, non-steroidal drugs, steroidal drugs, anti-bacterial drugs, water, and saline.

40. The method of claim 34 wherein applying comprises transmitting laser energy via a plurality of optical fiber terminal ends positioned near or at the distal end.

41. The method of claim 34 further comprising applying a cooling fluid onto the surface of the tissue region.

42. The method of claim 34 further comprising applying ultrasound energy to the tissue region via one or more ultrasound transducers positioned near or at the elongate shaft distal end.
